(19) United States
(12) Patent Application Publication
(10) Pub. No.: US 2025/0025699 A1
(43) Pub. Date: Jan. 23, 2025

(54) SYSTEMS AND METHODS WITH ACTIVATING RECHARGE STIMULATION

(71) Applicant: Boston Scientific Neuromodulation Corporation, Valencia, CA (US)

(72) Inventors: Brandon Thio, Durham, NC (US); Tianhe Zhang, Studio City, CA (US)

(21) Appl. No.: 18/775,287

(22) Filed: Jul. 17, 2024

Related U.S. Application Data
(60) Provisional application No. 63/527,722, filed on Jul. 19, 2023.

(51) Int. Cl. A61N 1/36

(52) U.S. Cl. CPC A61N 1/36178; A61N 1/36171; A61N 1/36175

(57) ABSTRACT

A neurostimulation system may include at least one electrode contact and a neurostimulator configured to use the at least one electrode contact to deliver a stimulation therapy to a patient using an electrical waveform that includes first phases of a first polarity and second phases of a second polarity opposite the first polarity. The neurostimulator may configured to deliver the stimulation therapy by therapeutically stimulating neural tissue using the both the first phases and the second phases of the electrical waveform. The second phases remove built up charge from the at least one electrode contact caused by the first phases, and the first phases remove built up charge from the at least one electrode contact caused by the second phases. Benefits include reducing energy consumption using the recharge pulse to activate a population of neural elements for reducing energy consumption.

### CLAIM OF PRIORITY

This application claims the benefit of U.S. Provisional Application No. 63/527,722 filed on Jul. 19, 2023, which is hereby incorporated by reference in its entirety.

### TECHNICAL FIELD

This document relates generally to medical systems, and more particularly, but not by way of limitation, to systems, devices, and methods for delivering neurostimulation.

### BACKGROUND

Neurostimulation has been proposed as a therapy for a number of conditions. Often, neurostimulation and neuromodulation may be used interchangeably to describe excitatory stimulation that causes action potentials as well as inhibitory and other effects. Examples of neuromodulation include Spinal Cord Stimulation (SCS), Deep Brain Stimulation (DBS), Peripheral Nerve Stimulation (PNS), and Functional Electrical Stimulation (FES).

The neurostimulation therapy may include an actively-driven phase (e.g., a stimulation pulse) followed by an active or passive recharge phase to recover charge injected during the actively-driven phase. The recharge phase that recovers charge is implemented to address potential electrode corrosion and potential tissue damage. A passive recharge phase may involve setting switches in the IPG to connect the electrodes to a common voltage, which effectively shorts the electrodes and causes any stored charge in the current paths to equilibrate by exponential decay through the patient's tissue. An active recharge phase forms part of a biphasic waveform that includes two opposite-polarity phases that are both actively driven with opposite polarity. The active recharge phase actively recovers charge that was injected during the first pulse phase. Specifically, charge is stored on capacitances in the current path when current is actively driven during the first pulse phase of a biphasic pulse, and stored charge is actively recovered and pulled off of those capacitances when the polarity and hence direction of the current is reversed during the second phase of the biphasic pulse. The interphase interval between the first and second phases of a conventional biphasic pulse is short (e.g., 100 μs) such that the second phase does not active neural elements.

However, as the recharge phase is actively driven in a biphasic pulse, such biphasic pulses require twice the amount of energy compared to equivalent monophasic pulses with passive recharge. Therefore, some fully implanted devices that have limited battery life may not implement biphasic pulses.

### SUMMARY

By way of example and not limitation, various embodiments provided herein provide a stimulation pulse paradigm that reduces energy consumption by delivering active recharge pulses in a manner that not only recovers charge but also activates neural elements. Thus, by way of example, a biphasic pulse delivered according to the present subject matter will stimulate neural elements using both phases of the biphasic pulse.

An example (e.g., Example 1) of a system may include a neurostimulation system including at least one electrode contact and a neurostimulator configured to use the at least one electrode contact to deliver a stimulation therapy to a patient using an electrical waveform that includes first phases of a first polarity and second phases of a second polarity opposite the first polarity. The neurostimulator may be configured to deliver the stimulation therapy by therapeutically stimulating neural tissue using both the first phases and the second phases of the electrical waveform. The second phases remove built up charge from the at least one electrode contact caused by the first phases, and the first phases remove built up charge from the at least one electrode contact caused by the second phases. The therapy may be provided by a cathodic phase and/or an anodic phase within the electrical waveform.

In Example 2, the subject matter of Example 1 may optionally be configured such that the electrical waveform includes a plurality of interphase intervals where each of the plurality of interphase intervals separate individual ones of the first phases and individual ones of the second phases. Interphase intervals may be longer than a refractory time of the neural tissue. However, faster-than-refractory stimulation may have desirable effects if purposely generating asynchronous activity in different neural elements.

In Example 3, the subject matter of Example 2 may optionally be configured such that the plurality of interphase intervals includes about an 11 ms interphase interval to stimulate the targeted neural tissue with a stimulation frequency of about 90 Hz.

In Example 4, the subject matter of any one or more of Examples 2-3 may optionally be configured such that the plurality of interphase intervals includes equal intervals.

In Example 5, the subject matter of any one or more of Examples 2-4 may optionally be configured such that the plurality of interphase intervals includes different intervals.

In Example 6, the subject matter of any one or more of Examples 2-5 may optionally be configured such that each of the plurality of interphase intervals includes at least 1 ms between successive ones of the first and second phases.

In Example 7, the subject matter of any one or more of Examples 2-5 may optionally be configured such that the electrical waveform includes a series of therapeutic pulses delivered during a window of time, the series of therapeutic pulses includes one or more pulses of the first phase and one or more pulses of the second phase for balancing a net charge on at least one of the first or the second electrode contacts in the window of time.

In Example 8, the subject matter of Example 7 may optionally be configured such that the neurostimulator is further configured to intermittently perform extra charge balancing in addition to the series of therapeutic pulses according to predefined charge balance rules to further balance the net charge in the window of time.

In Example 9, the subject matter of Example 8 may optionally be configured such that the predefined charge balance rules include rules for performing extra charge balancing at predefined times, after delivering a predefined number of therapeutic pulses, after delivering a predefined charge, after a predefined charge per unit of time, or based on an estimated instantaneous charge accounting for slow charge diffusion.

In Example 10, the subject matter of any one or more of Examples 8-9 may optionally be configured such that the neurostimulator is configured to perform extra charge balancing by inserting at least one non-therapeutic pulse to reduce a residual net charge within the window of time.

In Example 11, the subject matter of Example 10 may optionally be configured such that the therapeutic pulses have a first amplitude and a first pulse width and the non-therapeutic pulse has a second amplitude and a second pulse width, wherein the second amplitude is smaller than the first amplitude and is less than a depolarization threshold for the neural tissue, and the second pulse width is larger than the first pulse width.

In Example 12, the subject matter of Example 11 may optionally be configured such that the second amplitude is a fraction (1/X) of the first amplitude, the first pulse width is a fraction (1/Y) of the second pulse width (X and Y both being greater than 1), and Y is greater than X such that a charge provided by the non-therapeutic pulse is more than a charge provided by one of the therapeutic pulses. The charge per pulse corresponds to the area under the pulse curve (e.g., amplitude x pulse width for a rectilinear wave). The area under the curve for the non-therapeutic pulse may be larger than the area under the curve for the therapeutic pulse.

In Example 13, the subject matter of any one or more of Examples 8-12 may optionally be configured such that the neurostimulator is configured to perform extra charge balancing by monitoring a net charge and inserting charge to reduce the monitored net charge below the predefined threshold.

In Example 14, the subject matter of any one or more of Examples 1-13 may optionally be configured such that the electrical waveform is delivered using three or more electrode contacts, the first phases are distributed over at least one of the three or more electrode contacts, and the second phases are distributed over at least one of the three or more electrode contacts.

In Example 15, the subject matter of any one or more of Examples 1-14 may optionally be configured such that the electrical waveform is a first electrical waveform delivered over a first timing channel to a first set of electrode contacts that includes the at least one electrode contact. The neurostimulation system may be configured to deliver a second electrical waveform over a second timing channel to a second set of electrode contacts. The second electrical waveform may include first phases of a first polarity and second phases of a second polarity opposite the first polarity. At least one shared electrode contact may be in both the first and second sets of electrode contacts. At least one of the first and second phases of the second electrical waveform removes built up charge at the at least one shared electrode contact from the first electrical waveform.

Example 16 includes subject matter (such as a method, means for performing acts, machine readable medium including instructions that when performed by a machine cause the machine to perform acts, or an apparatus to perform). The subject matter may be used for delivering an electrical waveform using at least one electrode contact. The electrical waveform may include first phases of a first polarity and second phases of a second polarity opposite the first polarity. The delivering the electrical waveform may include therapeutically stimulating neural tissue using both the first phases and the second phases of the electrical waveform, and using the second phases to reduce built up charge from the at least one electrode contact caused by the first phases and using the first phases to reduce built up charge from the at least one electrode contact caused by the second phases.

In Example 17, the subject matter of Example 16 may optionally be configured such that the electrical waveform includes a plurality of interphase intervals, each of the plurality of interphase intervals separating individual ones of the first phases and individual ones of the second phases.

In Example 18, the subject matter of Example 17 may optionally be configured such that the plurality of interphase intervals includes about an 11 ms interphase interval to stimulate the targeted neural tissue with a stimulation frequency of about 90 Hz.

In Example 19, the subject matter of any one or more of Examples 17-18 may optionally be configured such that the plurality of interphase intervals includes equal intervals.

In Example 20, the subject matter of any one or more of Examples 17-19 may optionally be configured such that the plurality of interphase intervals includes different intervals.

In Example 21, the subject matter of any one or more of Examples 17-20 may optionally be configured such that each of the plurality of interphase intervals includes at least 1 ms between successive ones of the first and second phases.

In Example 22, the subject matter of any one or more of Examples 16-21 may optionally be configured such that the delivering the electrical waveform includes delivering a series of therapeutic pulses within a window of time by delivering both one or more pulses of the first phase and one or more pulses of the second phase for balancing a net charge on the at least one electrode contact in the window of time.

In Example 23, the subject matter of Example 22 may optionally be configured to further include intermittently performing extra charge balancing beyond the series of therapeutic pulses according to predefined charge balance rules.

In Example 24, the subject matter of Example 23 may optionally be configured such that the predefined charge balance rules include rules for performing extra charge balancing at predefined times, after delivering a predefined number of therapeutic pulses, after delivering a predefined charge, after a predefined charge per unit of time, or based on an estimated instantaneous charge accounting for slow charge diffusion.

In Example 25, the subject matter of any one or more of Examples 23-24 may optionally be configured such that the extra charge balancing is performed by inserting at least one non-therapeutic pulse to reduce a residual net charge within the window of time.

In Example 26, the subject matter of Example 25 may optionally be configured such that the therapeutic pulses have a first amplitude and a first pulse width and the non-therapeutic pulse has a second amplitude and a second pulse width. The second amplitude is smaller than the first amplitude and is less than a depolarization threshold for the neural tissue, and the second pulse width is larger than the first pulse width.

In Example 27, the subject matter of Example 26 may optionally be configured such that the second amplitude is a fraction (1/X) of the first amplitude, the first pulse width is a fraction (1/Y) of the second pulse width (X and Y both being greater than 1), and Y is greater than X such that a charge provided by the non-therapeutic pulse is more than a charge provided by one of the therapeutic pulses.

In Example 28, the subject matter of any one or more of Examples 23-27 may optionally be configured such that the neurostimulator is configured to perform extra charge balancing by monitoring a net charge and inserting charge to reduce the monitored net charge below the predefined threshold.

In Example 29, the subject matter of any one or more of Examples 16-28 may optionally be configured such that the delivering the electrical waveform includes using three or more electrode contacts, distributing the first phases over at least one of the three or more electrode contacts, and distributing the second phases over at least one of the three or more electrode contacts.

In Example 30, the subject matter of any one or more of Examples 16-29 may optionally be configured such that the delivering the electrical waveform includes delivering a first electrical waveform over a first timing channel to a first set of electrode contacts that includes the at least one electrical contact. The subject matter may further include delivering a second electrical waveform over a second timing channel to a second set of electrode contacts. The second electrical waveform may include first phases of a first polarity and second phases of a second polarity opposite the first polarity. At least one shared electrode contact is in both the first and second sets of electrode contacts. At least one of the first and second phases of the second electrical waveform removes built up charge at the at least one shared electrode contact from the first electrical waveform.

Example 31 includes subject matter (such as a non-transitory machine-readable medium including instructions, which when executed by a machine, cause the machine to perform a method for identifying effective placement of at least one lead having a plurality of electrodes) The method performed using the machine may include delivering an electrical waveform using at least one electrode contact. The electrical waveform may include first phases of a first polarity and second phases of a second polarity opposite the first polarity. The delivering the electrical waveform may include therapeutically stimulating neural tissue using both the first phases and the second phases of the electrical waveform, and using the second phases to reduce built up charge from the at least one electrode contact caused by the first phases and using the first phases to reduce built up charge from the at least one electrode contact caused by the second phases. In further examples, the subject matter of Example 32 may be configured such that the method performed by the machine may include any of the subject matter recited in Examples 17-30.

In Example 32, the subject matter of Example 31 may optionally be configured such that the electrical waveform includes a plurality of interphase intervals, and each of the plurality of interphase intervals separate individual ones of the first phases and individual ones of the second phases.

In Example 33, the subject matter of Example 32 may optionally be configured such that the plurality of interphase intervals includes about an 11 ms interphase interval to stimulate the targeted neural tissue with a stimulation frequency of about 90 Hz.

In Example 34, the subject matter of Example 31 may optionally be configured such that the method further includes intermittently performing extra charge balancing according to predefined charge balance rules.

In Example 35, the subject matter of Example 34 may optionally be configured such that the therapeutically stimulating neural tissue includes delivering therapeutic pulses having a first amplitude and a first pulse width, the performing extra charge balancing includes inserting a non-therapeutic pulse having a second amplitude and a second pulse width, the second amplitude is smaller than the first amplitude and is less than a depolarization threshold for the neural tissue, and the second pulse width is larger than the first pulse width, and a charge provided by the non-therapeutic pulse is more than a charge provided by one of the therapeutic pulses.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an embodiment of a neuromodulation system.

FIG. 2 illustrates an embodiment of a modulation device, such as may be implemented in the neuromodulation system of FIG. 1.

FIG. 3 illustrates an embodiment of a programming system such as a programming device, which may be implemented as the programming device in the neuromodulation system of FIG. 1.

FIG. 4 illustrates, by way of example, an implantable neuromodulation system and portions of an environment in which system may be used.

FIG. 5 illustrates, by way of example, an embodiment of a SCS system, which also may be referred to as a Spinal Cord Modulation (SCM) system.

FIG. 6 illustrates, by way of example and not limitation, an electrical waveform with first phases of a first polarity and second phases of a second polarity opposite the first polarity where both phases are used to therapeutically stimulate neural tissue while reducing built up charge.

FIG. 7 illustrates, by way of example and not limitation, a neurostimulator configured to deliver an electrical waveform over timing channels from a pulse generator to electrode contact sets.

FIGS. 8A-8B illustrate, by way of example and not limitation, current flow for a neurostimulation field generated by the first and second phases of the electrical waveform.

FIG. 9 illustrates, by way of example and not limitation, an electrical waveform with nonuniform biphasic pulses that have uniform interphase spacing between the first and second phases but have nonuniform intervals between biphasic pulses and nonuniform pulse amplitudes.

FIG. 10 illustrates, by way of example and not limitation, an electrical waveform with nonuniform biphasic pulses that have nonuniform interphase spacing between the first and second phases and have nonuniform intervals between biphasic pulses and nonuniform pulse amplitudes.

FIG. 11 illustrates, by way of example and not limitation, electrical waveforms delivered over multiple timing channels to different electrode contact sets in a manner to therapeutically stimulate neural tissue using both phases while reducing built up charge.

FIG. 12 illustrates, by way of example and not limitation, a method for delivering neurostimulation.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

The present subject matter provides systems, devices, and methods for delivering neurostimulation using electrical waveforms that use active recharge pulses to not only recover charge, but also to stimulate neural tissue. Therefore, in contrast to only using the second phase of a biphasic pulse to remove charge, both phases of a biphasic pulse may be used to deliver neurostimulation which may reduce energy consumption of the device used to deliver the neurostimulation.

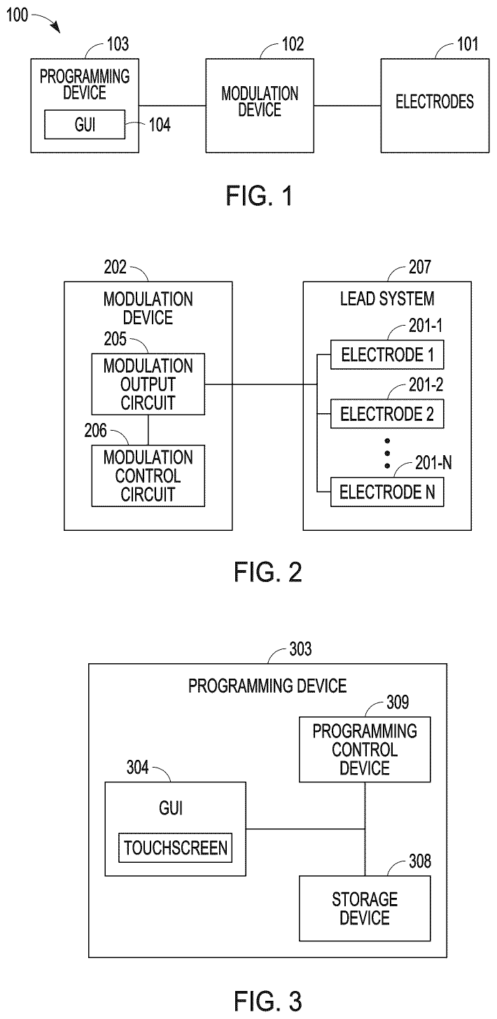

FIG. 1 illustrates, by way of example and not limitation, an embodiment of a neuromodulation system. The illustrated system 100 includes electrode contacts 101, which also may be simply referred to as electrodes, a modulation device 102, and a programming system such as a programming device 103. The programming system may include multiple devices. The electrode contacts 101 are configured to be placed on or near one or more neural targets in a patient. The modulation device 102 is configured to be electrically connected to electrode contacts 101 and deliver neuromodulation energy, such as in the form of electrical pulses, to the one or more neural targets though electrode contacts 101. The delivery of the neuromodulation is controlled by using a plurality of modulation parameters. The modulation parameters may specify the electrical waveform (e.g., pulses or pulse patterns or other waveform shapes) and a selection of electrode contacts through which the electrical waveform is delivered. In various embodiments, at least some parameters of the plurality of modulation parameters are programmable by a user, such as a physician or other caregiver. The programming device 103 provides the user with accessibility to the user-programmable parameters. In various embodiments, the programming device 103 is configured to be communicatively coupled to modulation device via a wired or wireless link. In various embodiments, the programming device 103 includes a graphical user interface (GUI) 104 that allows the user to set and/or adjust values of the user-programmable modulation parameters.

FIG. 2 illustrates an embodiment of a modulation device 202, such as may be implemented in the neuromodulation system 100 of FIG. 1. The modulation device 202 may also be referred to as a neurostimulator. Various embodiments of the neurostimulator may be used to deliver different types of neural therapy such as, but not limited to, SCS, DBS, PNS or FES therapy. The illustrated embodiment of the modulation device 202 includes a modulation output circuit 205 and a modulation control circuit 206. Those of ordinary skill in the art will understand that the neuromodulation system may include additional components such as sensing circuitry for patient monitoring and/or feedback control of the therapy, telemetry circuitry and power. The modulation output circuit 205 produces and delivers the neuromodulation. Neuromodulation pulses are provided herein as an example. However, the present subject matter is not limited to pulses, but may include other electrical waveforms (e.g., waveforms with different waveform shapes, and waveforms with various pulse patterns). The modulation control circuit 206 controls the delivery of the neuromodulation pulses using the plurality of modulation parameters. The lead system 207 includes one or more leads each configured to be electrically connected to modulation device 202 and a plurality of electrode contacts 201-1 to 201-N distributed in an electrode contact arrangement using the one or more leads. Each lead may have an electrode contact array consisting of two or more electrode contacts, which also may be referred to as contacts. Multiple leads may provide multiple electrode contact arrays to provide the electrode contact arrangement. Each electrode contact is a single electrically conductive contact providing for an electrical interface between modulation output circuit 205 and tissue of the patient, where N≥2. The neuromodulation pulses are each delivered from the modulation output circuit 205 through a set of electrode contacts selected from the electrode contacts 201-1 to 201-N. The number of leads and the number of electrode contacts on each lead may depend on, for example, the distribution of target(s) of the neuromodulation and the need for controlling the distribution of electric field at each target. In one embodiment, by way of example and not limitation, the lead system includes two leads each having eight electrode contacts. Some embodiments may use a lead system that includes a paddle lead.

The actual number and shape of leads and electrode contacts may vary for the intended application. An implantable waveform generator may include an outer case for housing the electronic and other components. The outer case may be composed of an electrically conductive, biocompatible material, such as titanium, that forms a hermetically-sealed compartment wherein the internal electronics are protected from the body tissue and fluids. In some cases, the outer case may serve as an electrode contact (e.g., case electrode). The waveform generator may include electronic components, such as a controller/processor (e.g., a microcontroller), memory, a battery, telemetry circuitry, monitoring circuitry, modulation output circuitry, and other suitable components known to those skilled in the art. The microcontroller executes a suitable program stored in memory, for directing and controlling the neuromodulation performed by the waveform generator. Electrical modulation energy is provided to the electrode contacts in accordance with a set of modulation parameters programmed into the pulse generator. By way of example but not limitation, the electrical modulation energy may be in the form of a pulsed electrical waveform. Such modulation parameters may comprise electrode contact combinations, which define the electrode contacts that are activated as anodes (positive), cathodes (negative), and turned off (zero), percentage of modulation energy assigned to each electrode contact (fractionalized electrode contact configurations), and electrical pulse parameters, which define the pulse amplitude (measured in milliamps or volts depending on whether the pulse generator supplies constant current or constant voltage to the electrode contact array), pulse width (measured in microseconds), pulse rate (measured in pulses per second), and burst rate (measured as the modulation on duration X and modulation off duration Y). Electrode contacts that are selected to transmit or receive electrical energy are referred to herein as “activated,” while electrode contacts that are not selected to transmit or receive electrical energy are referred to herein as “non-activated.”

Electrical modulation occurs between or among a plurality of activated electrode contacts, one of which may be the case of the waveform generator. The system may be capable of transmitting modulation energy to the tissue in a monopolar or multipolar (e.g., bipolar, tripolar, etc.) fashion. Monopolar modulation occurs when a selected one of the lead electrode contacts is activated along with the case of the waveform generator, so that modulation energy is transmitted between the selected electrode contact and case. Any of the electrode contacts E1 -E16 and the case electrode contact may be assigned to up to k possible groups or timing “channels.” In one embodiment, k may equal four. The timing channel identifies which electrode contacts are selected to synchronously source or sink current to create an electric field in the tissue to be stimulated. Amplitudes and polarities of electrode contacts on a channel may vary. In particular, the electrode contacts can be selected to be positive (anode, sourcing current), negative (cathode, sinking current), or off (no current) polarity in any of the k timing channels. The waveform generator may be operated in a mode to deliver electrical modulation energy that is therapeutically effective and causes the patient to perceive delivery of the energy (e.g., therapeutically effective to relieve pain with perceived paresthesia), and may be operated in a sub-perception mode to deliver electrical modulation energy that is therapeutically effective and does not cause the patient to perceive delivery of the energy (e.g., therapeutically effective to relieve pain without perceived paresthesia). The waveform generator may also be configured to deliver waveforms or pulses that are not therapeutically effective but are useful for intermittent charge balancing.

The waveform generator may be configured to individually control the magnitude of electrical current flowing through each of the electrode contacts. For example, a current generator may be configured to selectively generate individual current-regulated amplitudes from independent current sources for each electrode contact. In some embodiments, the pulse generator may have voltage regulated outputs. While individually programmable electrode contact amplitudes are desirable to achieve fine control, a single output source switched across electrode contacts may also be used, although with less fine control in programming. Neuromodulators may be designed with mixed current and voltage regulated devices.

The neuromodulation system may be configured to modulate spinal target tissue or other neural tissue. The configuration of electrode contacts used to deliver electrical pulses to the targeted tissue constitutes an electrode contact configuration, with the electrode contacts capable of being selectively programmed to act as anodes (positive), cathodes (negative), or left off (zero). In other words, an electrode contact configuration represents the polarity being positive, negative, or zero. An electrical waveform may be controlled or varied for delivery using electrode contact configuration(s). The electrical waveforms may be analog or digital signals. In some embodiments, the electrical waveform includes pulses. The pulses may be delivered in a regular, repeating pattern, or may be delivered using complex patterns of pulses that appear to be irregular. Other parameters that may be controlled or varied include the amplitude, pulse width, and rate (or frequency) of the electrical pulses. Each electrode contact configuration, along with the electrical pulse parameters, can be referred to as a “modulation parameter set.” Each set of modulation parameters, including fractionalized current distribution to the electrode contacts (as percentage cathodic current, percentage anodic current, or off), may be stored and combined into a modulation program that can then be used to modulate multiple regions within the patient.

The number of electrode contacts available combined with the ability to generate a variety of complex electrical waveforms (e.g., pulses), presents a huge selection of modulation parameter sets to the clinician or patient. For example, if the neuromodulation system to be programmed has sixteen electrode contacts, millions of modulation parameter sets may be available for programming into the neuromodulation system. Furthermore, for example SCS systems may have thirty-two electrode contacts which exponentially increases the number of modulation parameters sets available for programming. To facilitate such selection, the clinician generally programs the modulation parameters sets through a computerized programming system to allow the optimum modulation parameters to be determined based on patient feedback or other means and to subsequently program the desired modulation parameter sets.

FIG. 3 illustrates an embodiment of a programming system such as a programming device 303, which may be implemented as the programming device 103 in the neuromodulation system of FIG. 1. The programming device 303 includes a storage device 308, a programming control circuit 309, and a graphical user interface (GUI) 304. The programming control circuit 309 generates the plurality of modulation parameters that control the delivery of the neuromodulation pulses according to the pattern of the neuromodulation pulses. In various embodiments, the GUI 304 includes any type of presentation device, such as interactive or non-interactive screens, and any type of user input devices that allow the user to program the modulation parameters, such as touchscreen, keyboard, keypad, touchpad, trackball, joystick, and mouse. The storage device 308 may store, among other things, modulation parameters to be programmed into the modulation device. The programming device 303 may transmit the plurality of modulation parameters to the modulation device. In some embodiments, the programming device 303 may transmit power to the modulation device. The programming control circuit 309 may generate the plurality of modulation parameters. In various embodiments, the programming control circuit 309 may check values of the plurality of modulation parameters against safety rules to limit these values within constraints of the safety rules.

In various embodiments, circuits of neuromodulation, including its various embodiments discussed in this document, may be implemented using a combination of hardware, software and firmware. For example, the GUI circuit, modulation control circuit, and programming control circuit, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

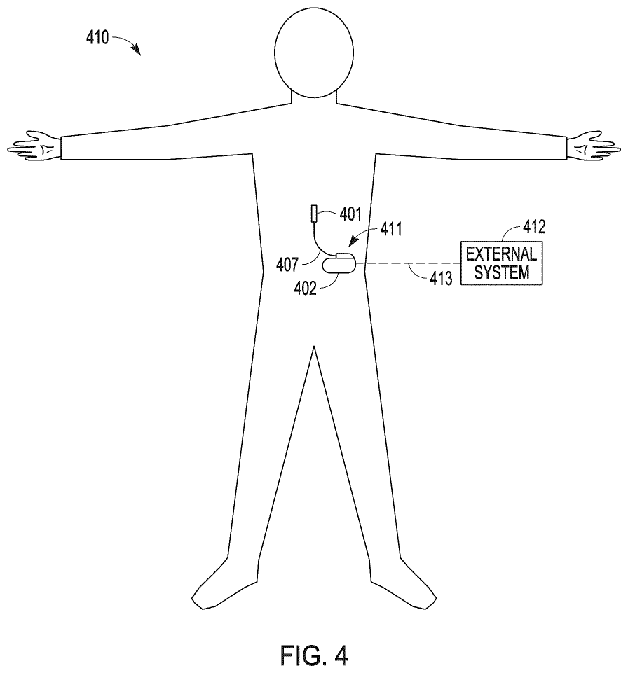

FIG. 4 illustrates, by way of example, an implantable neuromodulation system and portions of an environment in which system may be used. The system is illustrated for implantation near the spinal cord. However, neuromodulation system may be configured to modulate other neural targets including, but not limited to, SBS, PNS or FES targets. The system 410 includes an implantable system 411, an external system 412, and a telemetry link 413 providing for wireless communication between implantable system 411 and external system 412. The implantable system is illustrated as being implanted in the patient's body. The implantable system 411 includes an implantable modulation device (also referred to as an implantable pulse generator, or IPG) 402, a lead system 407, and electrode contacts 401. The lead system 407 includes one or more leads each configured to be electrically connected to the modulation device 402 and a plurality of electrode contacts 401 distributed in the one or more leads. In various embodiments, the external system 412 includes one or more external (non-implantable) devices each allowing a user (e.g., a clinician or other caregiver and/or the patient) to communicate with the implantable system 411. In some embodiments, the external system 412 includes a programming device intended for a clinician or other caregiver to initialize and adjust settings for the implantable system 411 and a remote control device intended for use by the patient. For example, the remote control device may allow the patient to turn a therapy on and off and/or adjust certain patient-programmable parameters of the plurality of modulation parameters. The external system 412 may include personal devices such as phones and tablets.

The neuromodulation lead(s) of the lead system 407 may be placed adjacent, i.e., resting near, or upon the dura, adjacent to the spinal cord area to be stimulated. For example, the neuromodulation lead(s) may be implanted along a longitudinal axis of the spinal cord of the patient. Due to the lack of space near the location where the neuromodulation lead(s) exit the spinal column, the implantable modulation device 402 may be implanted in a surgically-made pocket either in the abdomen or above the buttocks, or may be implanted in other locations of the patient's body. The lead extension(s) may be used to facilitate the implantation of the implantable modulation device 402 away from the exit point of the neuromodulation lead(s).

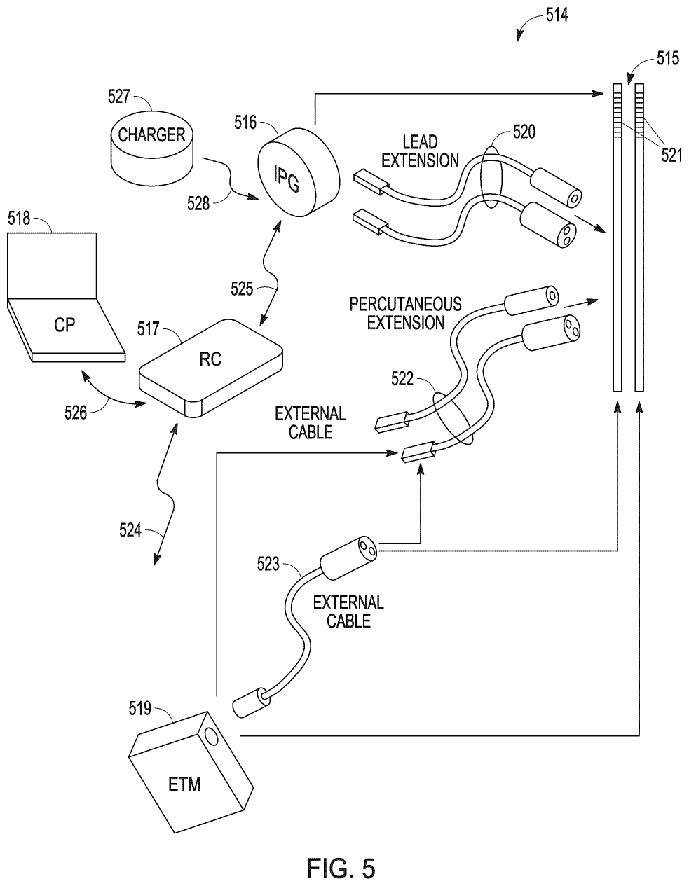

FIG. 5 illustrates, by way of example, an embodiment of a SCS system, which also may be referred to as a Spinal Cord Modulation (SCM) system. A similar system, with DBS lead(s), may be used to provide a DBS system. The SCS system 514 may generally include onc or more (illustrated as two) of implantable neuromodulation leads 515, an electrical waveform generator 516 such as an implantable pulse generator, an external remote controller (RC) 517, a clinician's programmer (CP) 518, and an external trial modulator (ETM) 519. IPGs are used herein as an example of the electrical waveform generator. However, it is expressly noted that the waveform generator may be configured to deliver regular, repeating patterns of pulses or in complex patterns that appear to be irregular patterns of pulses where pulses have differing amplitudes, pulse widths, pulse intervals, and bursts with differing number of pulses. It is also expressly noted that the waveform generator may be configured to deliver electrical waveforms other than pulses. The waveform generator 516 may include pulse generation circuitry that delivers electrical modulation energy in the form of a pulsed electrical waveform (i.e., a temporal series of electrical pulses) to the electrode contacts in accordance with a set of modulation parameters. The electrical waveform may include first phases of a first polarity and second phases of a second polarity opposite the first polarity. Neural tissue may be therapeutically stimulated using both the first phases and the second phases of the electrical waveform. The electrical waveform includes a plurality of interphase intervals, each of the plurality of interphase intervals separating individual ones of the first phases and individual ones of the second phases. The second phases may be used to reduce built up charge from the at least one electrode contact caused by the first phases and the first phases may be used to reduce built up charge from the at least one electrode contact caused by the second phases. The waveform generator 516 may be physically connected via one or more percutaneous lead extensions 520 to the neuromodulation leads 515, which carry a plurality of electrode contacts 521. As illustrated, the neuromodulation leads 515 may be percutaneous leads with the electrode contacts arranged in-line along the neuromodulation leads. Any suitable number of neuromodulation leads can be provided, including only one, as long as the number of electrode contacts is greater than two (including the waveform generator case function as a case electrode contact) to allow for lateral steering of the current. Alternatively, a surgical paddle lead can be used in place of one or more of the percutaneous leads.

The ETM 519 may also be physically connected via the percutaneous lead extensions 522 and external cable 523 to the neuromodulation leads 515. The ETM 519 may have similar waveform generation circuitry as the waveform generator 516 to deliver electrical modulation energy to the electrode contacts accordance with a set of modulation parameters. The ETM 519 is a non-implantable device that is used on a trial basis after the neuromodulation leads 515 have been implanted and prior to implantation of the waveform generator 516, to test the responsiveness of the modulation that is to be provided. Functions described herein with respect to the waveform generator 516 can likewise be performed with respect to the ETM 519.

The RC 517 may be used to telemetrically control the ETM 519 via a bi-directional RF communications link 524. The RC 517 may be used to telemetrically control the waveform generator 516 via a bi-directional RF communications link 525. Such control allows the waveform generator 516 to be turned on or off and to be programmed with different modulation parameter sets. The waveform generator 516 may also be operated to modify the programmed modulation parameters to actively control the characteristics of the electrical modulation energy output by the waveform generator 516. A clinician may use the CP 518 to program modulation parameters into the waveform generator 516 and ETM 519 in the operating room and in follow-up sessions. The waveform generator 516 may be implantable. The implantable waveform generator 516 and the ETM 519 may have similar features as discussed with respect to the modulation device 202 described with respect to FIG. 2.

The CP 518 may indirectly communicate with the waveform generator 516 or ETM 519, through the RC 517, via an IR communications link 526 or other link. The CP 518 may directly communicate with the waveform generator 516 or ETM 519 via an RF communications link or other link (not shown). The clinician detailed modulation parameters provided by the CP 518 may also be used to program the RC 517, so that the modulation parameters can be subsequently modified by operation of the RC 517 in a stand-alone mode (i.e., without the assistance of the CP 518). Various devices may function as the CP 518. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 518. Alternatively, such programming methodologies can be performed using firmware or hardware. In any event, the CP 518 may actively control the characteristics of the electrical modulation generated by the waveform generator 516 to allow the desired parameters to be determined based on patient feedback or other feedback and for subsequently programming the waveform generator 516 with the desired modulation parameters. To allow the user to perform these functions, the CP 518 may include a user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant waveform generator, implant waveform generator and lead(s), replace waveform generator, replace waveform generator and leads, replace or revise leads, explant, etc.), generate a pain map of the patient, define the configuration and orientation of the leads, initiate and control the electrical modulation energy output by the neuromodulation leads, and select and program the IPG with modulation parameters in both a surgical setting and a clinical setting.

An external charger 527 may be a portable device used to transcutaneously charge the waveform generator via a wireless link such as an inductive link 528. Once the waveform generator has been programmed, and its power source has been charged by the external charger or otherwise replenished, the waveform generator may function as programmed without the RC or CP being present.

Various embodiments of present subject matter provide an electrical waveform configured to provide a recharge phase of bipolar stimulation pulses. Bipolar stimulation consists of at least one electrode contact that functions as a cathode and at least one electrode contact that functions as an anode that are simultaneously on and current-balanced in space. Often, neural activation occurs near the cathode, and this cathodic neural activation is typically therapeutic. However, it is noted that there are times where the anodic phase can be therapeutic when the stimulation amplitude is high enough. For example, both phases are believed to be potentially therapeutic in deep brain stimulation.

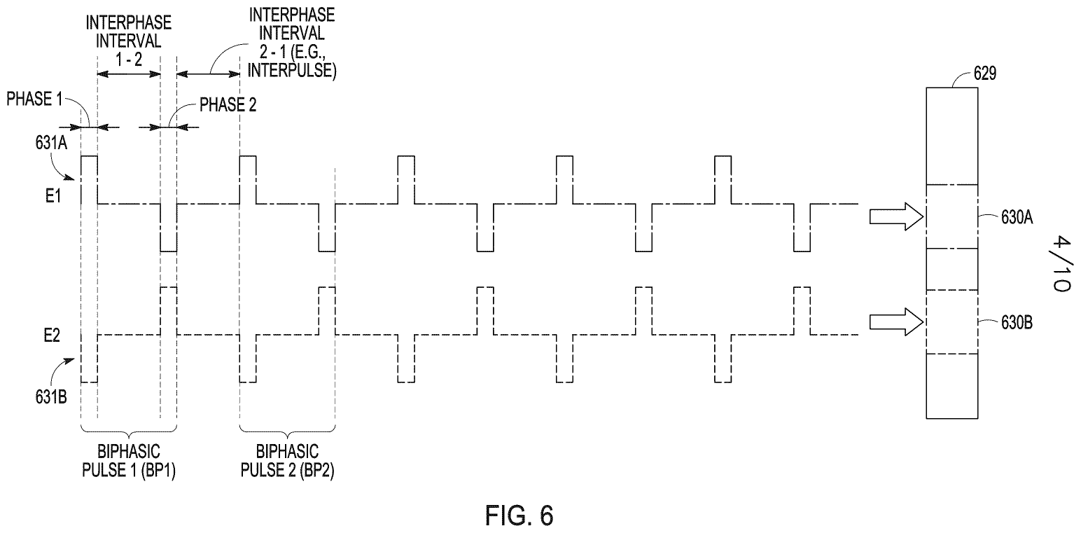

FIG. 6 illustrates, by way of example and not limitation, an electrical waveform with first phases of a first polarity and second phases of a second polarity opposite the first polarity where both phases are used to therapeutically stimulate neural tissue while reducing built up charge. The illustration shows a lead 629 with a first electrode contact 630A and a second electrode contact 630B used to deliver an electrical waveform. The waveform may be illustrated by the first waveform 631A at the first electrode contact 630A and by the second waveform 631B at the second electrode contact 630B. The pulses in the first waveform 630A and second waveform 630B are opposite as current flows out from the first electrode contact 630A will return into the second electrode contact 630B, and current flows from the second electrode contact 630B will return into the first electrode contact 630A.

The waveform is illustrated with alternating pulse phases (e.g., Phase 1 and opposite Phase 2), which together may be considered to be a biphasic pulse (e.g., see biphasic pulse 1 (BP1) and biphasic pulse 2 (BP2)). Neural populations may be activated during each of the two phases. The stimulated neural tissue may be in between the first and second electrode contacts, may be underneath either the first or second electrode contact, and/or may be an axon or other long conducting element that runs underneath both the first and second electrode contacts. For example, where the same neural tissue is activated by each of the two phases, desired stimulation frequency may be controlled using an interphase interval. In a specific example, a 10 ms interphase interval from pulse phase to pulse phase may be used to provide a 100 Hz (1/10 ms=100 Hz) stimulation frequency. In another specific example, an 11 ms interphase interval from pulse phase to pulse phase may be used to provide a 90 Hz (1/11 ms approximately equals 90 Hz) stimulation frequency. The second pulse, or recharge pulse, may be symmetric to the first pulse with opposite polarity. The second pulses may remove built up charge density on both contacts. For example, where the waveform is delivered using a first electrode contact and a second electrode contact, the first pulse may activate neural population at least near an original cathode and the second pulse may activate neural population at least near an original anode.

Some embodiments may intermittently introduce a charge balancing pulse or pulses to ensure that there is no charge accumulation on the electrode contacts generated by irreversible redox reactions. For example, some embodiments may incorporate a charge balancing pulse after every second stimulation pulse. The charge balancing pulse may “skip” cycles of the biphasic pulsing waveform and can take an arbitrary shape, including but not limited to rectangular, decaying exponential (encompassing interphase), triangular, rectified sinusoidal, and the like. The width and amplitude of the charge balancing pulse may depend on the waveform shape. The charge balance may be determined using the discrete integral or sum of the calculated charge accumulation from the stimulation phases. The charge balance “touch-up” may also account for charge bleed-off following initial stimulation phase, due to the length of the interphase between pulses. The extra charge balancing pulse may not activate neural elements and thus may be considered to be a non-therapeutic pulse rather than a therapeutic pulse. The extra charge balancing may be performed according to predefined charge balance rules. Examples of such rules include, but are not limited to, rules for performing extra charge balancing at predefined times, after delivering a predefined number of therapeutic pulses, after delivering a predefined charge, after a predefined charge per unit of time, or based on an estimated instantaneous charge accounting for slow charge diffusion. The non-therapeutic pulse may be delivered to reduce a residual net charge. The therapeutic pulses may have a first amplitude and a first pulse width and the non-therapeutic, charge balancing touch-up pulse may have a second amplitude and a second pulse width. Generally, the second amplitude may be smaller than the first amplitude and may be less than a depolarization threshold for the neural tissue. The second pulse width may be larger than the first pulse width. A charge provided by the non-therapeutic pulse may be more than a charge provided by one of the therapeutic pulses. For example, the charge associated with a rectilinear pulse is the area under the curve, which corresponds to the product of the pulse amplitude and the pulse width. The second amplitude for the non-therapeutic pulse may be a fraction (1/X) of the first amplitude for the therapeutic pulse (e.g., X being larger than 1). The first pulse width for the therapeutic pulse may be a fraction (1/Y) of the second pulse width of the non-therapeutic pulse (e.g., Y being larger than 1). That is, the proportion that the second pulse width is larger than the first pulse width is larger than the proportion that the first pulse amplitude is larger than the second pulse amplitude such that a charge provided by the non-therapeutic pulse is more than a charge provided by one of the therapeutic pulses. As identified above, the second amplitude may be smaller than the first amplitude and may be less than a depolarization threshold for the neural tissue, and the second pulse width may be larger than the first pulse width. However, if the first pulse is cathodic and the second pulse is anodic, charge balancing may be achieved using a technically larger anodic pulse without activating nearly as much underlying neural tissue, and the pulse width of the second pulse maybe correspondingly smaller. According to some embodiments, the neurostimulator may be configured to perform extra charge balancing by monitoring a net charge and inserting charge to reduce the monitored net charge below the predefined threshold.

The illustrated waveform in FIG. 6 is a relatively simple embodiment in which the each of the first and such pulse phases have the same pulse amplitude and the same pulse width, and further have the same pulse intervals including pulses intervals between the first phase and a subsequent second phase (interphase interval 1-2) and the same pulse intervals between the second phase and a subsequent first phase (interphase interval 2-1). Furthermore, the both interphase interval 1-2 and interphase interval 2-1 may be equal. However, the pulses do not need to be the same throughout the waveform so long as charge balance is maintained within a certain window and that the waveform remains globally unpolarized.

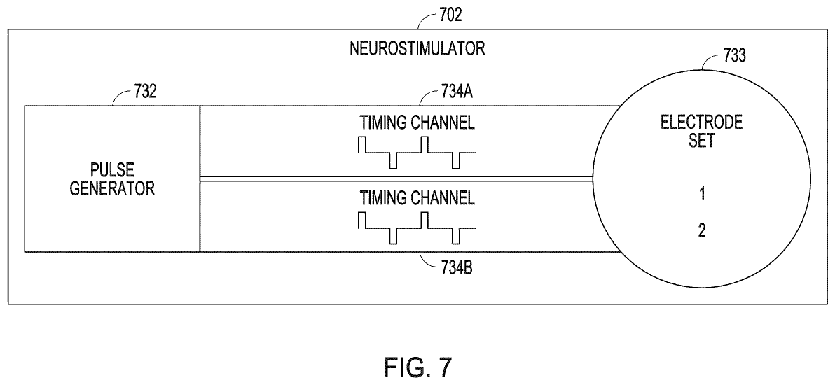

FIG. 7 illustrates, by way of example and not limitation, a neurostimulator configured to deliver an electrical waveform over timing channels from a pulse generator to electrode contact sets. The neurostimulator 702 may be an embodiment of the modulation device 102, 202 illustrated in FIGS. 1 and 2. The neurostimulator 702 includes a pulse generator 732, an arrangement of electrode contacts 733, and at least two channels 734A, 734B through which the pulse generator 732 delivers an electrical waveform to electrode contact sets 733 formed form the arrangement of electrode contacts. The waveform delivered in each timing channel 734A and 734B includes its own set of pulse parameters that define the electrical waveform, including pulse amplitude(s), pulse width(s), pulse-to-pulse interval(s), polarity, and the like. Further, each timing channel is connected to a set of electrode contacts, which may be different from the set of electrode contacts used by other timing channels. Thus, for example, the arrangement of electrode contacts may include a first set of electrode contacts and a second set of electrode contacts. The first and second sets may include one or more of the same electrode contacts, and the first and second sets may include one or more different electrode contacts. Furthermore, some of the electrode contact(s) in a given set may have the same first polarity and other electrode contact(s) in the given set may have the same second polarity opposite the first polarity.

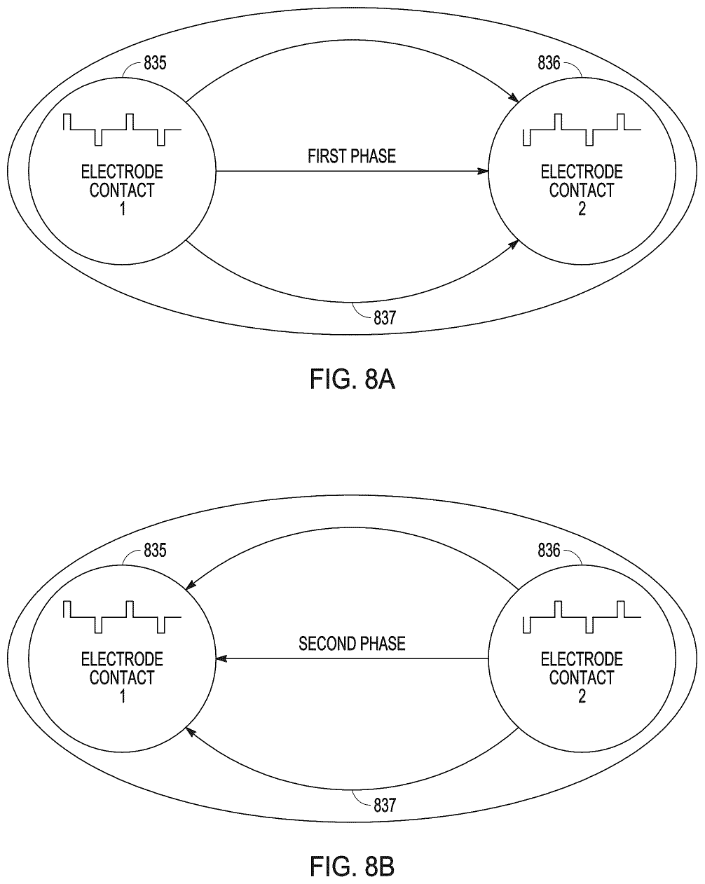

FIGS. 8A-8B illustrate, by way of example and not limitation, current flow for a neurostimulation field generated by the first and second phases of the electrical waveform. The field extends between or among the electrode contact(s) of a first subset 835 having the first polarity and the electrode contact(s) of a second subset 836 having the second polarity. For simplicity, each of the first and second subsets only include one electrode contact such that electrode contact 1 and electrode contact 2 have opposite polarities. Thus, during a first pulse phase as illustrated in FIG. 8A the stimulation field 837 may extend from electrode contact 1 where positive current is injected into the tissue to electrode contact 2 where positive current is returned; and during a second pulse phase as illustrated in FIG. 8B the stimulation field 837 may extend from electrode contact 2 where positive current is injected into the tissue to electrode contact 2 where positive current is returned. Those of ordinary skill in the neural stimulation art will understand that electrons, which are negatively charged, provide electron current in the opposite direction to the positive current. Thus, the first phase causes electrons to be present at electrode contact 2, and the second phase causes electrons to be present at electrode contact 1. Furthermore, the first phase tends to remove charge (e.g., electrons) that accumulated at electrode contact 1, and the second phase tends to remove charge (e.g., electrons) that accumulated at electrode contact 2. Often, neural activation occurs near the electrode contact(s) from which a negative pulse injects electron current into the tissue. These electrode contact(s) function as a cathode during the negative pulse. The negative (cathodic) pulse may be used to elicit action potentials in neural tissue and thus may be considered to be a therapeutic pulse. However, anodic stimulation from electrode contact(s) from which a positive pulse occurs also may provide therapeutic pulses.

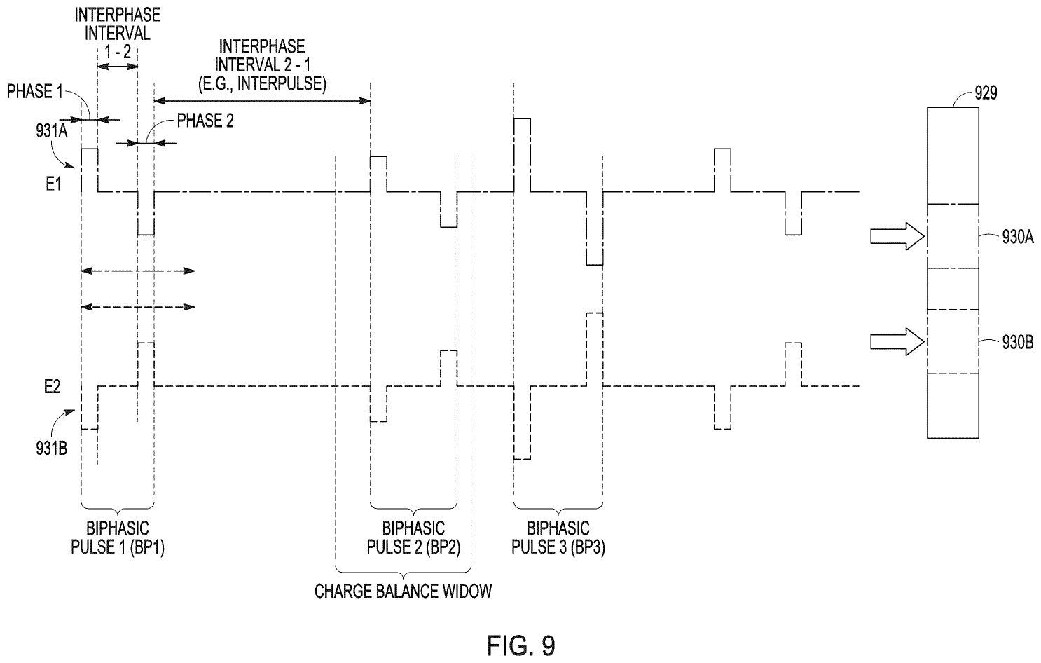

FIG. 9 illustrates, by way of example and not limitation, an electrical waveform with nonuniform biphasic pulses that have uniform interphase spacing between the first and second phases but have nonuniform intervals between biphasic pulses and nonuniform pulse amplitudes. The illustration shows a lead 929 with a first electrode contact 930A and a second electrode contact 930B used to deliver an electrical waveform. The waveform may be illustrated by the first waveform 931A at the first electrode contact 930A and by the second waveform 931B at the second electrode contact 930B. The pulses in the first waveform 930A and second waveform 930B are opposite as current flow out from the first electrode contact 930A will return into the second electrode contact 930B, and current flow from the second electrode contact 930B will return into the first electrode contact 930A. Each of the biphasic pulses (e.g., BP1, BP2, BP3, etc.) have first and second phases of equal magnitude but opposite polarity. Thus, each biphasic pulse is charge balanced. However, the interphase interval 1-2 from the first phase to the second phase is different for different ones of the biphasic pulses (e.g., BP1, BP2, BP3, etc.). Furthermore, the interphase interval 2-1 from the second phase to a subsequent first phase also varies in the electrical waveform. Where the electrical waveform includes a series of biphasic pulses, the interphase interval 2-1 may also be referred to as an interval between biphasic pulses or simply a biphasic pulse interval, and the interphase interval 1-2 may be referred to as an inter-biphasic pulse interval. The figure illustrates that individual cathodic and anodic pulses need not be spaced out evenly. However, the pulses may be delivered in a manner to maintain charge balance within a window of time. In some embodiments, the successive pulses may be charge balanced. Additionally, or alternatively, some embodiments may include charge balancing “touch-up” phases during interphase times.

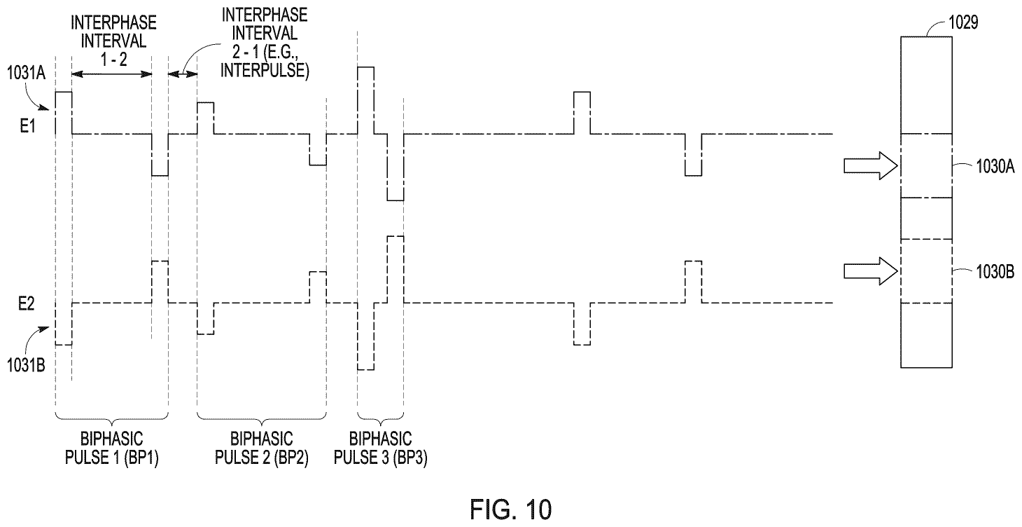

FIG. 10 illustrates, by way of example and not limitation, an electrical waveform with nonuniform biphasic pulses that have nonuniform interphase spacing between the first and second phases and have nonuniform intervals between biphasic pulses and nonuniform pulse amplitudes. The illustration shows a lead 1029 with a first electrode contact 1030A and a second electrode contact 1030B used to deliver an electrical waveform. The waveform may be illustrated by the first waveform 1031A at the first electrode contact 1030A and by the second waveform 1031B at the second electrode contact 1030B. The pulses in the first waveform 1030A and second waveform 1030B are opposite as current flow out from the first electrode contact 1030A will return into the second electrode contact 1030B, and current flow from the second electrode contact 1030B will return into the first electrode contact 1030A. Each of the biphasic pulses (e.g., BP1, BP2, BP3, etc.) have first and second phases of equal magnitude but opposite polarity. Thus, each biphasic pulse is charge balanced. However, the interphase interval 1-2 from the first phase to the second phase is different for different ones of the biphasic pulses (e.g., BP1, BP2, BP3, etc.). Additionally, the pulse amplitudes for one biphasic pulse may different from the pulse amplitudes for another biphasic pulse. Furthermore, the interphase interval 2-1 from the second phase to a subsequent first phase also varies in the electrical waveform. Where the electrical waveform includes a series of biphasic pulses, the interphase interval 2-1 may also be referred to as an interval between biphasic pulses or simply a biphasic pulse interval, and the interphase interval 1-2 may be referred to as an inter-biphasic pulse interval. The pulses may be delivered in a manner to maintain charge balance within a window of time. Some embodiments may include charge balancing “touch-up” phases during interphase times.

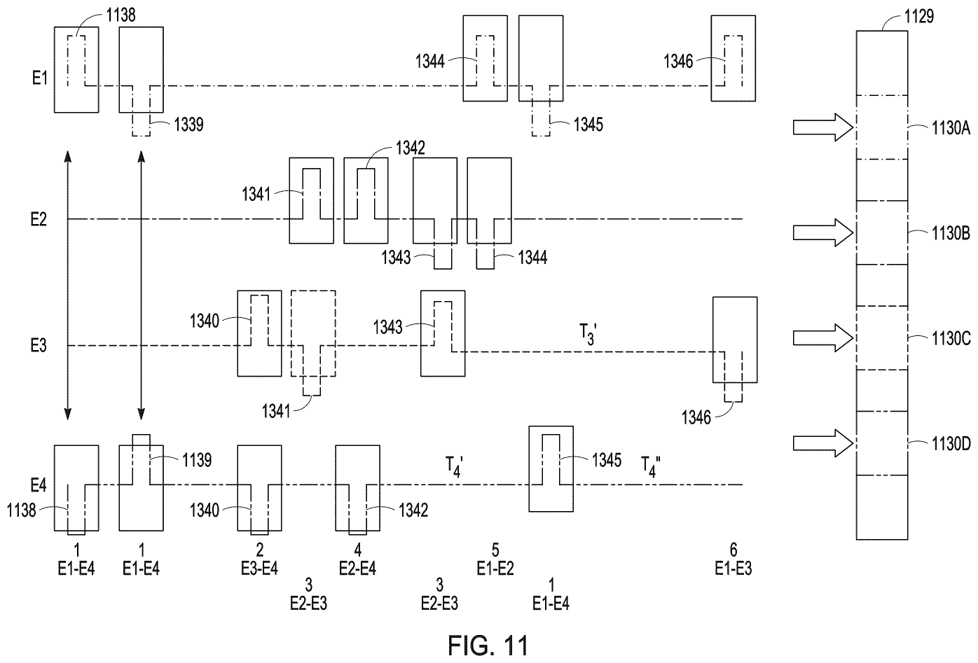

FIG. 11 illustrates, by way of example and not limitation, electrical waveforms delivered over multiple timing channels to different electrode contact sets in a manner to therapeutically stimulate neural tissue using both phases while reducing built up charge. The pulses may be delivered in a spatially distributed manner. That is, the pulses may be delivered using different combinations of electrode contacts. The charge balance may be maintained via touch up phases or through charge balance window rules as previously described. For simplicity, amplitudes are shown to be the same, and pulses are shown being delivered through two electrode contacts simultaneously. However, pulses could be spread across three or more electrode contacts in a manner that maintains charge balance rules. Some embodiments may implement rules such that stimulation phases are always alternating.

The illustration shows a lead 1129 with a first electrode contact 1130A, a second electrode contact 1130B, and third electrode contact 1130C and a fourth electrode contact 1130D used to deliver an electrical waveform. In the illustrated non-limiting example, the first pulse 1138 and the second pulse 1139 both use electrode contacts E1 and E4 (e.g., timing channel 1). The combinations of these two pulses are similar to the biphasic pulses illustrated in previous examples. However, the third pulse 1340 uses electrode contacts E3 and E4 (e.g., timing channel 2), the fourth pulse 1341 uses electrode contacts E2 and E3 (e.g., timing channel 3), the fifth pulse 1342 uses electrode contacts E2 and E4 (e.g., timing channel 4), the sixth pulse 1343 uses electrode contacts E2 and E3 (e.g., timing channel 3), the seventh pulse 1344 uses electrode contacts E1 and E2 (e.g., timing channel 5), the eighth pulse 1345 uses electrode contacts E1 and E4 (e.g., timing channel 1), and the ninth pulse 1346 uses electrode contacts E1 and E3 (e.g., timing channel 6). These pulses from different timing channels may cooperate to remove charge from previous pulses. Every contact may be paired or associated with at least one other electrode contact to deliver a pulse. Successive pulses of same polarity can be given in a manner to maintain charge balance within a time window. The interpulse interval may be defined to provide a desired stimulation frequency. For example, about an 11 ms interpulse interval may be used to provide a stimulation frequency of about 90 Hz. In an example, a range of frequencies from about 85 Hz to 95 Hz may be considered to be about 90 Hz. In an example, a tighter range of frequencies from about 88 Hz to 92 Hz may be considered to be about 90 Hz. In comparison, a standard interphase interval for a biphasic pulse is 100 μs, which is over a hundred times shorter. The pulse widths for the 90 Hz may be, by way of example and not limitation, within a range from 160 us to 260 μs. A waveform delivered according to these parameters are believed to be beneficial in quickly recruiting a larger area of neural tissue and providing fast-acting sub-perception therapy using energy-efficient, low frequencies. The waveform may also produce patterned (and not necessarily regular) activation of target dorsal column fibers that is sufficient to engage dorsal horn inhibitory mechanisms but not sufficient to cause paresthesias (e.g. via orthodromic activation of the dorsal column nuclei). The pattern is stimulation frequency-dependent, and simulations suggest that the patterns produced by a 90 Hz waveform result in effective inhibition (Gilbert, J. E., Titus, N., Zhang, T., Esteller, R., & Grill, W. M. (2022). Surround Inhibition Mediates Pain Relief by Low Amplitude Spinal Cord Stimulation: Modeling and Measurement. Eneuro).

As the interpulse intervals may reflect true stimulation frequencies, the interpulse intervals may be at least 1-2 ms and may range up to 1 second or greater durations. A 1 ms interpulse interval corresponds to about a 1 kHz signal and a 1 second interpulse interval corresponds to about a 1 Hz interval. Mandatory charge balance “touch ups” can be imposed according to charge balance rules. Charge balance rule examples include, but are not limited to, rules for performing extra charge balancing at predefined times, after delivering a predefined number of therapeutic pulses, after delivering a predefined charge, after a predefined charge per unit of time, or based on an estimated instantaneous charge accounting for slow charge diffusion. Touch ups can restore perfect charge balance or drop value to below a threshold. For example, the threshold may be derived from the g. Shannon model limit of 30μC/cm 2.

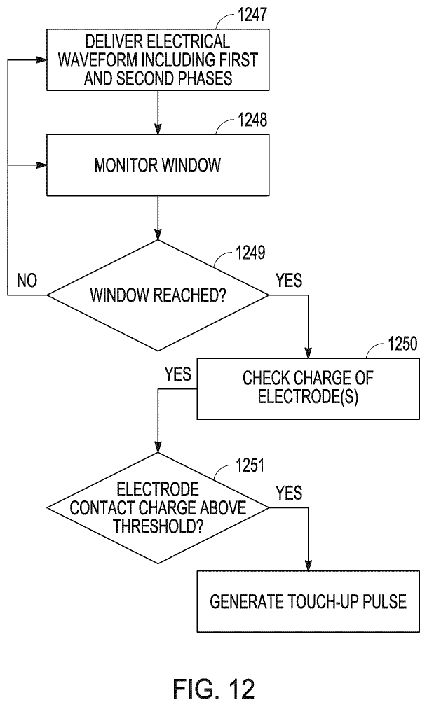

FIG. 12 illustrates, by way of example and not limitation, a method for delivering neurostimulation. The illustrated method may include, as illustrated at 1247, delivering an electrical waveform including first and second phases. At 1248, a window of time may be monitored for maintaining charge balance. A determination may be made at 1249 whether an end of the window has been reached. If the end has not been reached, the process may return to continuing to deliver electrical waveform 1247 and monitoring the window 1248. If an end of the window of time has been reached, the method may check the net charge of the electrode(s) 1250. For example, the charge may be sensed using the electrode contact(s) itself by sensing extracellular potential. At 1251, it may be determined whether the electrode contact charge is above a threshold. If it is not above the threshold, the process may return to continuing to deliver electrical waveform 1247 and monitoring the window 1248. If it is above the threshold, the process may proceed to generate a touchup pulse to further balance the charge 1252. The waveform may continue to be delivered by returning to 1247.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
delivering an electrical waveform using at least one electrode contact, wherein the electrical waveform includes first phases of a first polarity and second phases of a second polarity opposite the first polarity, wherein the delivering the electrical waveform includes therapeutically stimulating neural tissue using both the first phases and the second phases of the electrical waveform, and using the second phases to reduce built up charge from the at least one electrode contact caused by the first phases and using the first phases to reduce built up charge from the at least one electrode contact caused by the second phases.

2. The method of claim 1, wherein the electrical waveform includes a plurality of interphase intervals, each of the plurality of interphase intervals separating individual ones of the first phases and individual ones of the second phases.

3. The method of claim 2, wherein the plurality of interphase intervals includes about an 11 ms interphase interval to stimulate the targeted neural tissue with a stimulation frequency of about 90 Hz.

4. The method of claim 2, wherein the plurality of interphase intervals includes equal intervals.

5. The method of claim 2, wherein the plurality of interphase intervals includes different intervals.

6. The method of claim 2, wherein each of the plurality of interphase intervals includes at least 1 ms between successive ones of the first and second phases.

7. The method of claim 1, wherein the delivering the electrical waveform includes delivering a series of therapeutic pulses within a window of time by delivering both one or more pulses of the first phase and one or more pulses of the second phase for balancing a net charge on the at least one electrode contact in the window of time.

8. The method of claim 7, further comprising intermittently performing extra charge balancing beyond the series of therapeutic pulses according to predefined charge balance rules.

9. The method of claim 8, wherein the predefined charge balance rules include rules for performing extra charge balancing at predefined times, after delivering a predefined number of therapeutic pulses, after delivering a predefined charge, after a predefined charge per unit of time, or based on an estimated instantaneous charge accounting for slow charge diffusion.

10. The method of claim 8, wherein the extra charge balancing is performed by inserting at least one non-therapeutic pulse to reduce a residual net charge within the window of time.

11. The method of claim 10, wherein the therapeutic pulses have a first amplitude and a first pulse width and the non-therapeutic pulse has a second amplitude and a second pulse width, wherein the second amplitude is smaller than the first amplitude and is less than a depolarization threshold for the neural tissue, and the second pulse width is larger than the first pulse width.

12. The method of claim 11, wherein a proportion that the second pulse width is larger than the first pulse width is larger than a proportion that the first pulse amplitude is larger than the second pulse amplitude such that a charge provided by the non-therapeutic pulse is more than a charge provided by one of the therapeutic pulses such that a charge provided by the non-therapeutic pulse is more than a charge provided by one of the therapeutic pulses.

13. The method of claim 8, wherein the neurostimulator is configured to perform extra charge balancing by monitoring a net charge and inserting charge to reduce the monitored net charge below the predefined threshold.

14. The method of claim 1, wherein the delivering the electrical waveform includes using three or more electrode contacts, distributing the first phases over at least one of the three or more electrode contacts, and distributing the second phases over at least one of the three or more electrode contacts.

15. The method of claim 1, wherein the delivering the electrical waveform includes delivering a first electrical waveform over a first timing channel to a first set of electrode contacts that includes the at least one electrical contact, the method further including delivering a second electrical waveform over a second timing channel to a second set of electrode contacts, wherein the second electrical waveform includes first phases of a first polarity and second phases of a second polarity opposite the first polarity, and wherein at least one shared electrode contact is in both the first and second sets of electrode contacts, wherein at least one of the first and second phases of the second electrical waveform removes built up charge at the at least one shared electrode contact from the first electrical waveform.

16. A non-transitory machine-readable medium including instructions, which when executed by a machine, cause the machine to perform a method comprising:
delivering an electrical waveform using at least one electrode contact, wherein the electrical waveform includes first phases of a first polarity and second phases of a second polarity opposite the first polarity, wherein the delivering the electrical waveform includes therapeutically stimulating neural tissue using both the first phases and the second phases of the electrical waveform, and using the second phases to reduce built up charge from the at least one electrode contact caused by the first phases and using the first phases to reduce built up charge from the at least one electrode contact caused by the second phases.

17. A system, comprising:
a neurostimulation system including at least one electrode contact and a neurostimulator configured to use the at least one electrode contact to deliver a stimulation therapy to a patient using an electrical waveform that includes first phases of a first polarity and second phases of a second polarity opposite the first polarity, wherein the neurostimulator is configured to deliver the stimulation therapy by therapeutically stimulating neural tissue using the both the first phases and the second phases of the electrical waveform, the second phases remove built up charge from the at least one electrode contact caused by the first phases, and the first phases remove built up charge from the at least one electrode contact caused by the second phases.

18. The system of claim 17, wherein the electrical waveform includes a plurality of interphase intervals, each of the plurality of interphase intervals separating individual ones of the first phases and individual ones of the second phases.

19. The system of claim 18, wherein the plurality of interphase intervals includes about an 11 ms interphase interval to stimulate the targeted neural tissue with a stimulation frequency of about 90 Hz.

20. The system of claim 17, wherein:
the electrical waveform includes a series of therapeutic pulses delivered during a window of time;
the series of therapeutic pulses include one or more pulses of the first phase and one or more pulses of the second phase for balancing a net charge on at least one of the first or the second electrode contacts in the window of time;
the neurostimulator is further configured to intermittently perform extra charge balancing in addition to the series of therapeutic pulses according to predefined charge balance rules to further balance the net charge in the window of time;
the neurostimulator is configured to perform extra charge balancing by inserting at least one non-therapeutic pulse to reduce a residual net charge within the window of time;
the therapeutic pulses have a first amplitude and a first pulse width and the non-therapeutic pulse has a second amplitude and a second pulse width;
the second amplitude is smaller than the first amplitude and is less than a depolarization threshold for the neural tissue; and
the second pulse width is larger than the first pulse width.
